(19) United States
(12) Patent Application Publication
(10) Pub. No.: US 2010/0049117 A1
(43) Pub. Date: Feb. 25, 2010

(54) Microporator for Porating a Biological Membrane and Integrated Permeant Administering System

(75) Inventors: Thomas Bragagna, Feldkirch (AT); Reinhard Braun, Lustenau (AT); Daniel Gfrerer, Bludenz (AT); Bernhard Nussbaumer, Feldkirch (AT)

Correspondence Address: FISH & ASSOCIATES, PC;ROBERT D. FISH, 2603 Main Street, Suite 1000, Irvine, CA, 92614-6232, US

(73) Assignee: PANTEC BIOSOLUTIONS AG, Ruggell (LI)

(21) Appl. No.: 11/911,859

(22) PCT Filed: Apr. 18, 2005
(86) PCT No.: PCT/US05/51702

§ 371 Date: Oct. 18, 2007

(51) Int. Cl. A61N 1/30

(52) U.S. Cl. 604/20

(57) ABSTRACT

A micro-porator (10) for porating a biological membrane (1), comprising: a) a controller (11) b) an initial microporation dataset (D); c) and an ablator (10a) for creating a microporation on the biological membrane (1), the controller (11) controlling the ablator (10a) based on the initial microporation dataset (D), to create the microporation as defined by the initial microporation dataset (D).

### FIELD OF THE INVENTION

This invention relates generally to the field of transmembrane delivery of permeants like drugs or bioactive molecules to an organism. More particularly, this invention relates to a microporator for porating a biological membrane and an integrated permeant administering system comprising the microporator for porating the biological member.

### BACKGROUND OF THE INVENTION

Many new drugs, including vaccines, proteins, peptides and DNA constituents, have been developed for better and more efficient treatment for disease and illness. Especially due to recent advances in molecular biology and biotechnology, increasingly potent pharmaceutical agents, such as recombinant human insulin, growth hormone, follicle stimulating hormone, parathyroid hormone, etanercept, and erythropoietin are available. However, one significant limitation in using these new drugs is often a lack of an efficient drug delivery system, especially where the drug needs to be transported across one or more biological barriers at therapeutically effective rates and amounts.

Among other things, currently known methods and devices fail to allow controlled and reproducible administration of drugs. Currently known methods and devices also fail to provide prompt initiation and cut-off of drug delivery with improved safety, efficiency and convenience. It is therefore an object of the present invention to provide devices and methods to improve transmembrane delivery of molecules, permeants including drugs and biological molecules, across biological membranes, such as tissue or cell membranes. This problem is solved with a laser microporator comprising the features of claim 1. Dependent claims 2 to 10 disclose optional features. The problem is further solved with an integrated permeant administering system comprising the features of claim 11, with dependent claims 12 to 18 disclosing optional features. The problem is further solved with a method for operating the micro-porator comprising the features of claim 21. The problem is further solved with a method for administering a permeant comprising the features of claim 22.

### SUMMARY OF THE INVENTION

The device and method according to the invention utilize a micro-porator for porating a biological membrane like the skin, to create a microporation consisting of a plurality of individual pores. In a preferred embodiment a laser micro-porator is used. The micro-porator ablates or punctures the biological membrane, in particular the stratum corneum and part of the epidermis of the skin. This affects individual micropores in the skin, which results in an increase in skin permeability to various substances, which allows a transdermal or intradermal delivery of substances applied onto the skin. A microporation created by the micro-porator in one session comprises a plurality of individual pores, having a total number in the range between 10 and 1 million individual pores. By each individual pore a permeation surface within the skin is created. Depending on the number and shape of the individual pores an initial permeation surface is created, which is the sum of the permeation surfaces of all individual pores. Due to cell growth, the permeation surface of each individual pore decreases over time, and therefore also the total permeation surface, which is the sum of the permeation surface of all individual pores, decreases over time. The decrease of the permeation surface over time depends in particular on the geometrical shape of the individual pore. By an appropriate choice of the number of individual pores and their shape, not only the initial permeation surface but also the decrease of the total permeation surface over time can be determined. The appropriate choice of number and shape can be calculated and stored as an initial microporation dataset. The micro-porator according to the invention has the ability to reproducibly create a microporation with a predetermined initial permeation surface and preferably also with a predetermined function of the total permeation surface over time. Any biological tissue, but in particular the skin, can be porated with a microporator according to the invention.

Various techniques can be used for creating pores in biological tissues. Preferably a microporator using a laser beam for creating pores is used. But, for example, also a device for heating via conductive materials or a device generating high voltage electrical pulses can be used for creating pores. U.S. Pat. No. 6,148,232, for example, disclose a technique for creating micro-channels by using an electrical field. This device could also be suitable for creating micropores of predetermined shapes, if provided with additional means to reproducibly create micropores, such as feedback means according to the invention, to detect characteristics of the individual micropores.

The amount of substances delivered through the biological membrane, in particular from the surface of the skin to within the human body, depends on the permeation surface and its variation over time. The present invention therefore also provides an integrated permeant administering system to provide a permeant like a drug, to provide an appropriate initial microporation dataset, and to provide a micro-porator to create a microporation according to the individual microporation dataset. After the microporation is created, a permeant is applied onto the skin, and the transdermal or intradermal delivery of the permeant takes place in a predetermined way. To apply the permeant effectively, it is important to fit properties of the permeant and the microporation accordingly, to ensure a desired local or systemic effect, for example to ensure a predetermined concentration of a drug in the blood.

According to one preferred embodiment, the system allows, for a specific drug, to select an appropriate initial microporation dataset out of a plurality of initial microporation datasets, so that a microporation is created according to the appropriate initial microporation dataset. When the respective drug is applied onto the skin, the transdermal delivery of the drug in function of time is mainly determined by the function of the permeation surface over time. The integrated permeant administering system therefore also allows to individually apply a drug, and for example to reach a predetermined concentration of a drug in the blood according to individual needs.

As used herein, “poration” or “microporation” means the formation of small holes or pores to a desired depth in or through the biological membrane or tissue, such as the skin, the mucous membrane or an organ of a human being or a mammal, or the outer layer of an organism or a plant, to lessen the barrier properties of this biological membrane to the passage of permeants or drugs into the body. The microporation referred to herein shall be no smaller than 1 micron across and at least 1 micron in depth.

As used herein, “micropore”, “pore” or “Individual pore” means an opening formed by the microporation method.

As used herein “ablation” means the controlled removal of material which may include cells or other components comprising some portion of a biological membrane or tissue. The ablation can be caused, for example, by one of the following:
kinetic energy released when some or all of the vaporizable components of such material have been heated to the point that vaporization occurs and the resulting rapid expansion of volume due to this phase change causes this material, and possibly some adjacent material, to be removed from the ablation site;
Thermal or mechanical decomposition of some or all off the tissue at the poration site by creating a plasma at the poration site;
heating via conductive materials;
high voltage AC current;
pulsed high voltage DC current;
micro abrasion using micro particles;
pressurised fluid (air, liquid);
pyrotehnic;
Electron beam or ion beam;
The device causing the ablation is herein called the ablator.

As used herein, “tissue” means any component of an organism including but not limited to, cells, biological membranes, bone, collagen, fluids and the like comprising some portion of the organism.

As used herein “puncture” or “micro-puncture” means the use of mechanical, hydraulic, sonic, electromagnetic, or thermal means to perforate wholly or partially a biological membrane such as the skin or mucosal layers of a human being or a mammal, or the outer tissue layers of a plant.

To the extent that “ablation” and “puncture” accomplish the same purpose of poration, i.e. the creating a hole or pore in the biological membrane optionally without significant damage to the underlying tissues, these terms may be used interchangeably.

As used herein “puncture surface” means the surface of the hole or pore at the outer surface of the biological membrane, which has been ablated or punctured.

As used herein the terms “transdermal” or “percutaneous” or “transmembrane” or “transmucosal” or “transbuccal” or “transtissual” or “intratissual” means passage of a permeant into or through the biological membrane or tissue to deliver permeants intended to affect subcutaneous layers and further tissues such as muscles, bones. In the most preferred embodiment the transdermal delivery introduces permeants into the blood, to achieve effective therapeutic blood levels of a drug.

As used herein the term “intradermal” means passage of a permeant into or through the biological membrane or tissue to delivery the permeant to the dermal layer, to therein achieve effective therapeutic or cosmetic tissue levels of a permeant, or to store an amount of permeant during a certain time in the biological membrane or tissue, for example to treat conditions of the dermal layers beneath the stratum corneum.

As used herein, “permeation surface” means the surface of the tissue surrounding the micropore or pore. “Permeation surface” may mean the surface of an individual micropore or pore, or may mean the total permeation surface, which means the sum of all individual surfaces of all individual micropores or pores.

As used herein, “corrected permeation surface” means the permeation surface corrected by a factor or a specific amount, for example by subtracting the surface of the micropore or pore which is part of the stratum corneum.

As used herein, the term “bioactive agent,” “permeant,” “drug,” or “pharmacologically active agent” or “deliverable substance” or any other similar term means any chemical or biological material or compound suitable for delivery by the methods previously known in the art and/or by the methods taught in the present invention, that induces a desired effect, such as a biological or pharmacological effect, which may include but is not limited to (1) having a prophylactic effect on the organism and preventing an undesired biological effect such as preventing an infection, (2) alleviating a lack or excess of substances (e.g. vitamins, electrolytes, etc.), (3) alleviating a condition caused by a disease, for example, alleviating pain or inflammation caused as a result of disease, (4) either alleviating, reducing, or completely eliminating the disease from the organism, and/or (5) the placement within the viable tissue layers of the organism of a compound or formulation which can react, optionally in a reversible manner, to changes in the concentration of a particular analyte and in so doing cause a detectable shift in this compound or formulation's measurable response to the application of energy to this area which may be electromagnetic, mechanical or acoustic. The effect may be local, such as providing for a local anaesthetic effect, or it may be systemic. This invention is not drawn to novel permeants or to new classes of active agents other than by virtue of the microporation technique, although substances not typically being used for transdermal, transmucosal, transmembrane or transbuccal delivery may now be useable. Rather it is directed to the mode of delivery of bioactive agents or permeants that exist in the art or that may later be established as active agents and that are suitable for delivery by the present invention.

Such substances include broad classes of compounds normally delivered into the organism, including through body surfaces and membranes, including skin as well as by injection, including needle, hydraulic, or hypervelocity methods. In general, this includes but is not limited to: Polypeptides, including proteins and peptides (e.g., insulin); releasing factors, including luteinizing Hormone Releasing Hormone (LHRH); carbohydrates (e.g., heparin); nucleic acids; vaccines; and pharmacologically active agents such as antiinfectives such as antibiotics and antiviral agents; analgesics and analgesic combinations; anorexics; antihelminthics; antiarthritics; antiasthmatic agents; anticonvulsants; antidepressants; antidiabetic agents; antidiarrheals; antihistamines; antiinflammatory agents; antimigraine preparations; antinauseants; antineoplastics; antiparkinsonism drugs; antipruritics; antipsychotics; antipyretics; antispasmodics; anticholinergics; sympathomimetics; xanthine derivatives; cardiovascular preparations including potassium and calcium channel blockers, beta-blockers, alpha-blockers, and antiarrhythmics; antihypertensives; diuretics and antidiuretics; vasodilators including general coronary, peripheral and cerebral; central nervous system stimulants; vasoconstrictors; cough and cold preparations, including decongestants; hormones such as estradiol, testosterone, progesterone and other steroids and derivatives and analogs, including corticosteroids; hypnotics; immunosuppressives; muscle relaxants; parasympatholytics; psychostimulants; sedatives; and tranquilizers. By the method of the present invention, both ionized and nonionized permeants may be delivered, as can permeants of any molecular weight including substances with molecular weights ranging from less than 10 Daltons to greater than 1,000,000 Daltons.

As used herein, an “effective” amount of a permeant means a sufficient amount of a compound to provide the desired local or systemic effect and performance at a reasonable benefit/risk ratio attending any treatment.

As used herein, “carriers” or “vehicles” refer to carrier materials without significant pharmacological activity at the quantities used that are suitable for administration with other permeants, and include any such materials known in the art, e.g., any liquid, gel, solvent, liquid diluent, solubilizer, microspheres, ilposomes, microparticles, lipid complexes, or the like, that is sufficiently nontoxic at the quantities employed and does not interact with the drug to be administered in a deleterious manner. Examples of suitable carriers for use herein include water, buffers, mineral oil, silicone, inorganic or organic gels, aqueous emulsions, liquid sugars, lipids, microparticles and nanoparticles, waxes, petroleum jelly, and a variety of other oils, polymeric materials and liposomes.

As used herein, a “biological membrane” means a tissue material present within a living organism that separates one area of the organism from another and, in many instances, that separates the organism from its outer environment. Skin and mucous and buccal membranes are thus included as well as the outer layers of a plant. Also, the walls of a cell, organ, tooth, bone, or a blood vessel would be included within this definition.

As used herein, “transdermal flux rate” is the rate of passage of any bioactive agent, drug, pharmacologically active agent, dye, particle or pigment in and through the skin separating the organism from its outer environment. “Transmembrane flux rate” refers to such passage through any biological membrane.

The term “individual pore” as used in the context of the present application refers to a micropore or a pore, in general a pathway extending from the biological membrane. The biological membrane for example being the skin, the individual pore then extending from the surface of the skin through all or significant part of the stratum corneum. In the most preferred embodiment the pathway of the individual pore extending through all the stratum corneum and part of the epidermis but not extending into the dermis, so that no bleeding occurs. In the most preferred embodiment the individual pore having a depth between 10 μm (for newborns 5 μm) and 150 μm.

As used herein the term “initial microporation” refers to the total number of pores created. “Initial microporation dataset” refers to the set of data, wherein the initial microporation is defined. The dataset including at least one parameter selected from the group consisting of: cross-section, depth, shape, permeation surface, total number of individual pores, geometrical arrangement of the pores on the biological membrane, minimal distance between the pores and total permeation surface of all individual pores. Preferably the initial microporation dataset defines the shape and geometrical arrangement of all individual pores, which then will be created using the microporator, so that the thereby created initial microporation is exactly defined and can be reproduced on various locations of the biological membrane, also on different objects subjects or persons.

The present invention employs a micro-porator comprising a controller, an initial microporation dataset and an ablator for creating a microporation, the controller reading the initial microporation dataset, and the controller controlling the ablator based on the initial microporation dataset to create a microporation as defined by the initial microporation dataset. Thereby a microporation is created with a predetermined Initial permeation surface, and preferably also with a predetermined permeation surface over time.

The ablator can be built in various ways, using various techniques. The ablator can for example consist of mechanically driven needles. The needles may be heated to ablate the biological membrane by heating. In the most preferred embodiment a pulsed laser beam is used to create individual pores. In a preferred embodiment, the laser micro-porator applies a parallel or quasi-parallel laser beam on the biological membrane, which facilitates control over the precise shape of the individual pore. The term “parallel or quasi-parallel laser beam” used herein refers to a laser beam that has a divergence of less than 3°, at least within a certain range of focus, the focus or focus range, extending in direction of the propagation direction of the laser beam, is a range of about 1 cm to 5 cm, preferably a range of 2 cm to 3 cm. The laser micro-porator using a parallel or quasi-parallel laser beam, allows creation of individual pores with highly reproducible permeation surfaces. In the most preferred embodiment the laser micro-porator comprises a feedback loop which is operatively coupled to the poration controller that actuates the laser source. The poration controller compares the measured characteristic of an individual pore with a predetermined value and stops emitting further laser pulses on the individual pore if the characteristic of the individual pore corresponds to the preset value, or if the characteristic of the individual pore 2 is within a preset range. Most preferred the depth of the individual pore is monitored. This allows creation of an individual pore similar to drilling a hole in a material, in that the depth of the hole e.g. the pore is repeatedly measured. This allows to very accurately microporate a biological membrane so that the created microporation corresponds to the predetermined values of the initial microporation dataset.

The plurality of laser pulses applied onto the same pore allows creating individual pores having a reproducible shape of the wall surrounding the individual pore and preferably allows also creating a reproducible shape of the lower end of the individual pore. The surface of the wall and the lower end is of importance, in particular the sum of the surface of the wall and the surface of the lower end which are part of the epidermis or the dermis, because this sum of surfaces forms a permeation surface through which most of the permeate passes into the tissue, for example into the epidermis and the dermis.

In a further embodiment the micro-porator is able to detect the depth at which the stratum corneum ends, e.g. the epidermis starts, for example, by using a spectrograph. This allows measuring the thickness of the stratum corneum and for example altering the total depth of created pores. With the initial microporation dataset, also the final depth of each individual pore may be defined. This final depth can now be corrected in that the thickness of the stratum corneum is added. The individual pore is then created with this corrected depth, which means the individual pore becomes deeper, and which means that the permeation surface of the epidermis corresponds to the given permeation surface. This is of importance, because the transdermal flux rate, depending on the drug applied, often depends on the size of permeation surface which allows a high passage of drugs, which might be the permeation surface of the epidermis only.

If the depth of the individual pore is not corrected by the thickness of the stratum corneum, the effect of the stratum corneum can be considered by calculating a corrected permeation surface. This corrected permeation surface for example comprising only the permeation surface of the epidermis. The total permeation surface of all individual pores can also be determined. Knowing the corrected permeation surface, which means the permeation surface of the epidermis, allows one to better control or predict the transdermal delivery of drug into the patient, e.g. to better control or predict the release of the drug into the patient.

The micro-porator can create a microporation having a number of individual pores in the range between 10 and up to 1 million, and having individual pores with a width between 0.01 and 0.5 mm, and a depth between 5 μm and 200 μm as defined by the initial microporation dataset.

In a preferred embodiment the micro-porator comprises an interface to at least read the initial microporation dataset, and to preferably read further parameters like permeant information, user information or porator application information. In a further preferred embodiment the micro-porator comprises a database that stores a plurality of initial microporation datasets. In a further preferred embodiment the micro-porator comprises a selector, which manually or automatically selects, for example based on user information such as the age, the most appropriate initial microporation dataset. The pores are then created according to this most appropriate initial microporation dataset.

The micro-porator can also comprise an inhibitor which inhibits the porator from porating if certain conditions are not fulfilled.

The micro-porator according to the invention allows creating on a biological membrane a wide variety of different, reproducible microporations, such as a wide variety of initial permeation surfaces and such as a wide variety of decreases of the permeation surface over time. The permeation surface affects the transdermal or intradermal delivery of the permeant like the drug. Therefore even the same drug or the same amount of drug applied onto the skin can be delivered differently into the skin, depending on the permeation surface. According to the invention an integrated permeant administering system is proposed, which considers relevant parameters regarding the permeant, the initial microporation dataset and the micro-porator, so that, after microporating the skin and after applying the drug onto the skin, the drug is released as requested into the skin, so that, for example, a defined blood-level profile is achieved.

After the poration is completed, a substance such as a drug is applied onto the skin, preferably in form of a transdermal patch. The transdermal patch offers a variety of significant clinical benefits over other dosage forms. Because passive as well as active transdermal patches deliver a predetermined drug concentration, and because the permeation surface over time being known, the transdermal patch offers controlled release of the drug into the patient, which for example enables a defined blood-level profile, resulting in reduced systemic side effects and, sometimes, improved efficacy over other dosage forms. In addition, transdermal patches are user-friendly, convenient, painless, and offer multi-day dosing. Transdermal patches therefore offer improved patient compliance. A substance can also be applied for cosmetic purpose only, for example applied intradermal.

The micro-porator for porating a biological membrane may be designed, for example, as the laser micro-porator disclosed in PCT patent application No. PCT/EP2005/051704 of the same applicant, filed on the same day and entitled “Laser microporator and method for operating a laser microporator”. The biological membrane may be porated according to a method, for example, as disclosed in PCT patent application No. PCT/EP2005/051703 of the same applicant, filed on the same day and entitled “Method for creating a permeation surface”. All citations herein are incorporated by reference in their entirety.

### BRIEF DESCRIPTION OF THE DRAWINGS

The present invention can be better understood and its advantages appreciated by those skilled in the art by referencing to the accompanying drawings, which are incorporated herein by reference. Although the drawings illustrate certain details of certain embodiments, the invention disclosed herein is not limited to only the embodiments so illustrated. Unless otherwise apparent from the context, all ranges include the endpoints thereof.

FIG. 1 shows a schematic cross-section of one pore of a laser porated skin;

FIG. 2 shows a laser micro-porator device;

FIG. 2a shows a further micro-porator device;

FIG. 3a shows a perspective view of a micro-poration of the skin;

FIG. 3b shows a plan view of the skin with an array of micro-porations;

FIG. 3c shows a schematic cross-section of a porated skin with a drug container attached to the skin surface;

FIG. 4 shows the permeation surface of all micropores over time;

FIG. 5a shows a given permeation surface and a created permeation surface;

FIG. 5b shows transdermal delivery of a drug over time, in combination with a permeation surface;

FIG. 6 shows a drug cassette containing two drug containers;

FIG. 7 shows a schematic view of a laser micro-porator;

FIG. 8 shows a schematic view of a further laser micro-porator;

FIG. 9 shows a block diagram of an integrated drug administering system;

FIG. 10a, 10b show the serum concentration of a drug over time, with the same amount of drug but different permeation surfaces.

### DETAILED DESCRIPTION

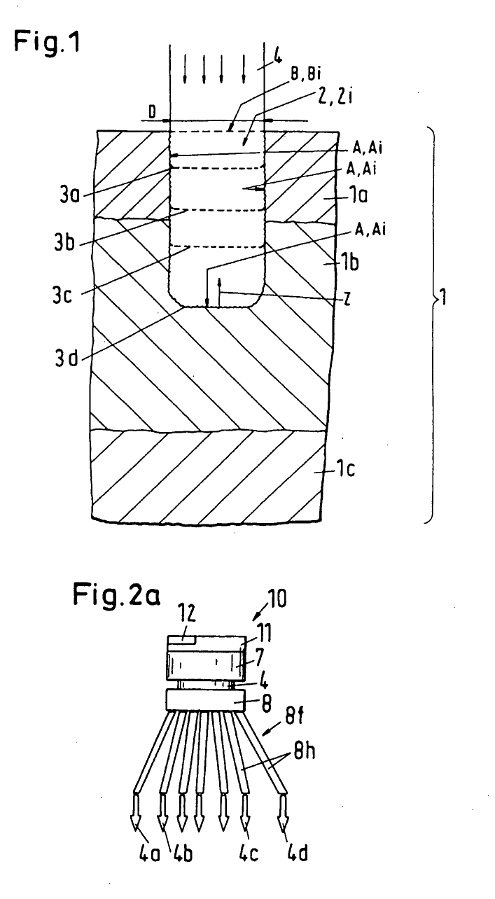

FIG. 1 shows a cross-sectional view of the top layers of the biological membrane 1, a human skin, including a stratum corneum 1a, an epidermal layer or epidermis 1b and a dermal layer or dermis 1c. Underlying the stratum corneum 1a is the viable epidermis or epidermal layer 1b, which usually is between 50 and 150 μm thick. The epidermis contains no blood vessels and freely exchanges metabolites by diffusion to and from the dermis 1c, located immediately below the epidermis 1b. The dermis 1c is between 1 and 3 mm thick and contains blood vessels, lymphatics and nerves. Once a drug reaches the dermal layer, the drug will generally perfuse through system circulation.

FIG. 1 also shows a parallel or quasi-parallel laser beam 4 having a circular shape with a diameter D and acting on the surface of the skin 1. The impact of the laser beam 4 onto the skin 1 causes an ablation of the tissue. A first shot of the laser beam 4 causes an individual pore 2 with a lower end 3a. The first shot effecting a puncture surface B at the outer surface of the skin 1 in the size of about (D/2)²*p, which corresponds to the amount of the outer surface of the biological membrane, which has been ablated or punctured. A second shot of the laser beam 4 at the same location causes an increase in depth of the individual pore 2 up to the lower end 3b, and a third and forth shot at the same location causes a further increase in depth up to the lower ends 3c and 3d. The total surface of the tissue 1 surrounding the individual pore 2 corresponds to the permeation surface A. There is no tissue 1 at the puncture surface B, therefore the puncture surface B is not part of the permeation surface A.

Each individual pore 2 of the epidermis 1b has a cell growth of usually (untreated) 3 to 15 μm per day, the cells usually growing from the lower end of the individual pore 2 in direction Z to the stratum corneum 1a. Which means the lower end 3d of the individual pore 2 is moving into the direction of the stratum corneum with a speed of about 3 to 15 μm/day, thereby reducing the permeation surface A. The corrected permeation surface, being the permeation surface of the epidermis 1b only, without the surface of the stratum corneum 1a, becomes the size of the puncture surface, which means the surface of the hole in the stratum corneum, as soon as the cells have reached the stratum corneum 1a. The remaining hole in the stratum corneum 1a will by the time be filled by death cells of the epidermis, which significantly increases the barrier properties in the remaining hole, and which regenerates the stratum corneum. At the end the individual pore 2 has vanished due to cell growth, and the formerly ablated tissue is regenerated by new cells.

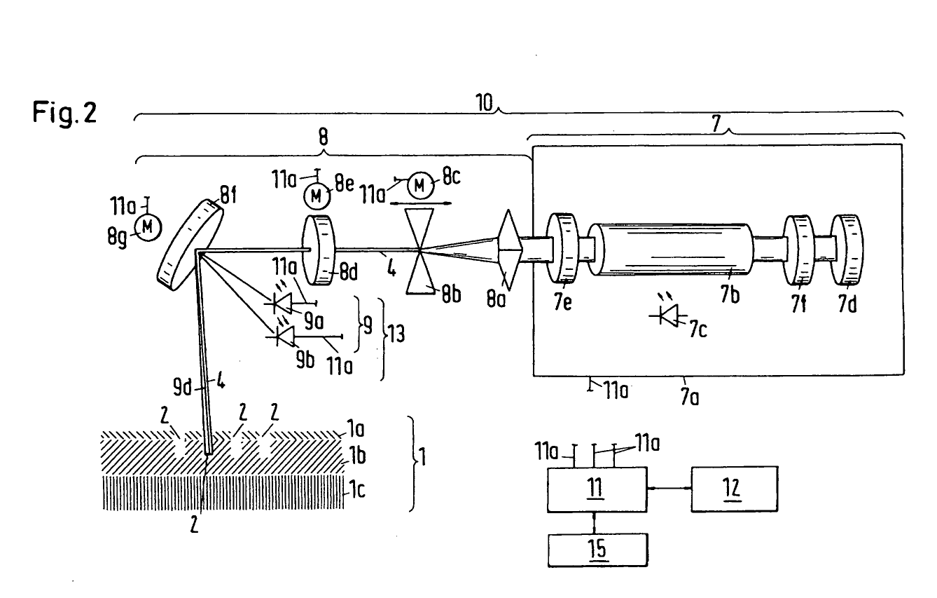

FIG. 2 shows a laser micro-porator 10 comprising a laser source 7 and a laser beam shaping and guiding device 8. The laser source 7 comprises a laser pump cavity 7a containing a laser rod 7b, preferably Er doped YAG, an exciter 7c that excites the laser rod 7b, an optical resonator comprised of a high reflectance mirror 7d positioned posterior to the laser rod and an output coupling mirror 7e positioned anterior to the laser rod, and an absorber 7f positioned posterior to the laser rod. A focusing lens 8a and a concave diverging lens 8b are positioned beyond the output coupling mirror 7e, to create a parallel or quasi-parallel laser beam 4. The diverging lens 8b can be moved by a motor 8c in the indicated direction. This allows a broadening or narrowing of the laser beam 4, which allows changing the width of the laser beam 4 and the energy fluence of the laser beam 4. A variable absorber 8d, driven by a motor 8e, is positioned beyond the diverging lens 8b, to vary the energy fluence of the laser beam 4. A deflector 8f, a mirror, driven by an x-y-drive 8g, is positioned beyond the absorber 8d for directing the laser beam 4 in various directions, to create individual pores 2 on the skin 1 on different positions. The laser microporator 10 also comprises a control device 11, which connected by wires 11a with the laser source 7, drive elements 8c, 8e, 8g, sensors and other elements not disclosed in detail.

In a preferred embodiment the laser porator 10 also includes a feedback loop. In FIG. 2, the feedback loop comprises an apparatus 9 to measure the depth of the individual pore 2, and preferably includes a sender 9a with optics that produce a laser beam 9d, and a receiver with optics 9b. The laser beam 9d has a smaller width than the diameter of the individual pore 2, for example five times smaller, so that the laser beam 9d can reach the lower end of the individual pore 2. The deflection mirror 8f directs the beam of the sender 9a to the individual pore 2 to be measured, and guides the reflected beam 9d back to the receiver 9b. In a preferred embodiment, the depth of the individual pore 2 is measured each time after a pulsed laser beam 4 has been emitted to the individual pore 2, allowing controlling the effect of each laser pulse onto the depth of the individual pore 2. The feedback loop 13 may, for example, comprise a sender 9a and a receiver 9b, built as a spectrograph 14, to detect changes in the spectrum of the light reflected by the lower end of the individual pore 2. This allows, for example, detecting whether the actual lower end 3a, 3b, 3c, 3d of the individual pore 2 is part of the stratum corneum 1a or of the epidermis 1b. The controller 11 also comprises a poration memory 12 containing at least specific data of the individual pores 2, in particular the initial microporation dataset. The laser porator 10 preferably creates the individual pores 2 as predescribed in the poration memory 12. The laser porator 10 also comprises one ore more input-output device 15 or interfaces 15, to enable data exchange with the porator 10, for example to enable the transfer of the parameters of the individual pores 2, the initial microporation dataset, into the poration memory 12, or to get data such as the actual depth or the total surface Ai of a specific individual pore 2i.

The pulse repetition frequency of the laser source 7 is within a range of 1 Hz to 1 MHz, preferably within 100 Hz to 100 kHz, and most preferred within 500 Hz to 10 kHz. Within one application of the laser porator 10, between 2 and 1 million individual pores 2 can be produced in the biological membrane 1, preferably 2 to 10000 individual pores 2, and most preferred 10 to 1000 individual pores 2, each pore 2 having a width in the range between 0.05 mm and 0.5 mm, and each pore 2 having a depth in the range between 5 μm and maximal 150 μm, but the lower end of the individual pore 2 being within the epidermis 1b.

The laser porator 10 also comprises an interlock mechanism, so that a laser pulse is emitted only when it is directed onto the skin 1.

In a preferred embodiment the feedback loop 9 is operatively coupled to the poration controller 11, which, for example, can compare the depth of the individual pore 2 with a predetermined value, so that no further pulse of the laser beam 4 is directed to the individual pore 2 if the characteristic of the individual pore 2, for example, the depth, is greater than or equal to a preset value, or if the characteristic of the individual pore 2 is within a predetermined range. This allows creation of individual pores 2 with a predetermined depth in a further embodiment, the laser beam 4 is operated as follows: If, for example, the measured depth is close to the value of the predetermined depth, the emitted energy per pulse of the laser beam 4 can be reduced, to create a pulse that ablates a smaller amount of tissue per pulse, so that the final depth of the individual pore 2 can be reached more accurate.

FIG. 2a shows a further embodiment of a laser micro-porator 10 comprising a controller 11, a single laser source 7 and optics 8 which guide the laser beam 4 into a plurality of fiberoptics 8h, thereby splitting up the laser beam 4 into a plurality of individual laser beams 4a, 4b, 4c, 4d. All fiberoptics 8h together form a deflector 8f, which directs the individual laser beams 4a, 4b, 4c, 4d in various directions. The exit end of each fiberoptics 8h has an individually oriented surface, such that the individual laser beams 4a, 4b, 4c, 4d leaving the fiberoptics 8h form an array of, for example, parallel individual laser beams 4a, 4b, 4c, 4d. The controller 11 comprises a poration memory 12, wherein at least an initial microporation dataset D can be stored.

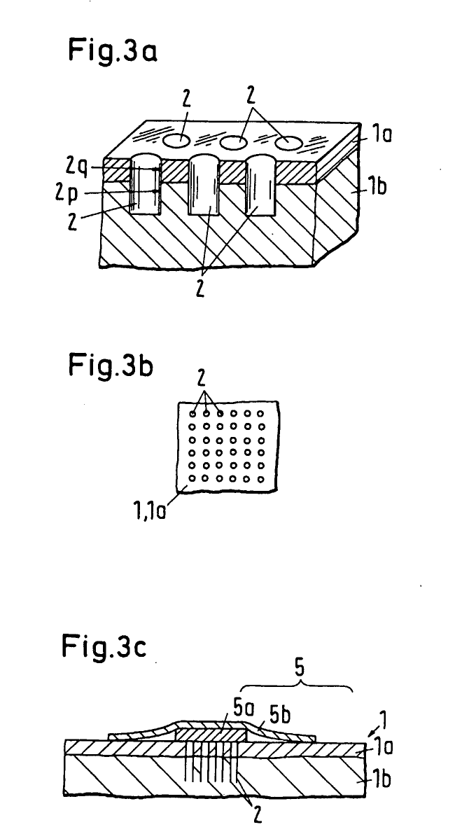

FIG. 3a shows an array of individual pores 2 in the skin 1, created by a micro-porator 10. In this example, all individual pores 2 have about the same shape and depth. The individual pores 2 may also have different shapes and depths, depending on the initial microporation dataset D.

FIG. 3b shows a plan view of the skin having a regular array of individual pores 2 that collectively form a micro-poration. The micro-poration on the biological membrane, after the laser porator 10 has finished porating, is called “initial microporation”. The poration memory 12 contains the initial microporation dataset, which define the initial microporation. The initial microporation dataset comprises any suitable parameters, including: width, depth and shape of each pore, total number of individual pores 2, geometrical arrangement of the pores 2 on the biological membrane, minimal distance between the pores 2, and so forth. The laser porator 10 creates the pores 2 as defined by the initial microporation dataset D. This also allows arranging the individual pores 2 in various shapes on the skin 1.

FIG. 3c discloses a transdermal patch 5 comprising a drug container 5a and an attachment 5b, which is attached onto the skin 1, the drug container 5a being positioned above an area comprising individual pores 2. The area can have a surface, depending on the number and spacing of the individual pores 2, in the range between 1 mm² and 1600 mm². Preferred 20×20 mm, e.g. a surface of 400 mm².

For each individual pore 2i, the surface of the inner wall and the surface of the lower end are of importance, in particular the permeation surface Ai, being the sum of both of these surfaces. In a preferred embodiment, the laser porator 10 comprises a distance measurement apparatus 9, which facilitates determining the permeation surface Ai very accurately. In a further preferred embodiment, the beginning of the epidermis is estimated by first determining the thickness of the stratum corneum. This in turn either permits determination of a corrected permeation surface Ai for each individual pore 2i, which establishes the effective permeation surface of the epidermis 1b, or which permits to increase the depth of the individual pore 2i by the thickness of the stratum corneum. This permeation surface Ai can easily be calculated for each individual pore 2i. If the individual pore 2i has the shape of, for example, a cylinder, the permeation surface Ai corresponds to the sum of D*p*H and (D/2)²*p, D being the diameter of the individual pore 2, and H being the total depth of the individual pore 2 or the depth of the individual pore 2 within the epidermis 1b. The effective permeation surface Ai in the pore 2 often doesn't correspond exactly to the geometrical shape, defined by D and H because the surface of the pore 2 may be rough or may comprise artefacts, which means the effective permeation surface is bigger than the calculated permeation surface Ai. The permeation surface Ai is at least a reasonable estimate of the effective permeation surface. Usually there is only a small or no difference between the permeation surface Ai and the effective permeation surface in the pore 2. The total permeation surface A of n individual pores 2i is then the sum A of all permeation surfaces Ai of all individual pores 2i.

Each individual pore 2 of the epidermis has a cell growth of usually 3 to 15 μm per day, the cells growing from the lower end of the individual pore 2 in direction Z to the stratum corneum 1a. This cell growth causes the permeation surface Ai of each individual pore 2i, respectively the total permeation surface A of all individual pores 2 to decrease in function of time. Depending on the total number of individual pores 2, which can be in a range of up to 100 or 1000 or 10000 or even more, the geometrical shape of the individual pores 2, and taking into account the effect of cell growth, the total permeation surface in function of time can be varied in a wide range. The initial permeation surface and also the decrease of the permeation surface over time can be predicted and calculated by an appropriate choice of the number of pores 2 and their geometrical shape. This definition of all pores is stored as the initial microporation dataset D. Correction factors may be applied to this initial microporation dataset D, for example based on user information like individual speed of cell growth, or based on the optional use of regeneration delayer like occlusive bandage, diverse chemical substances, etc., which influence the speed of cell growth.

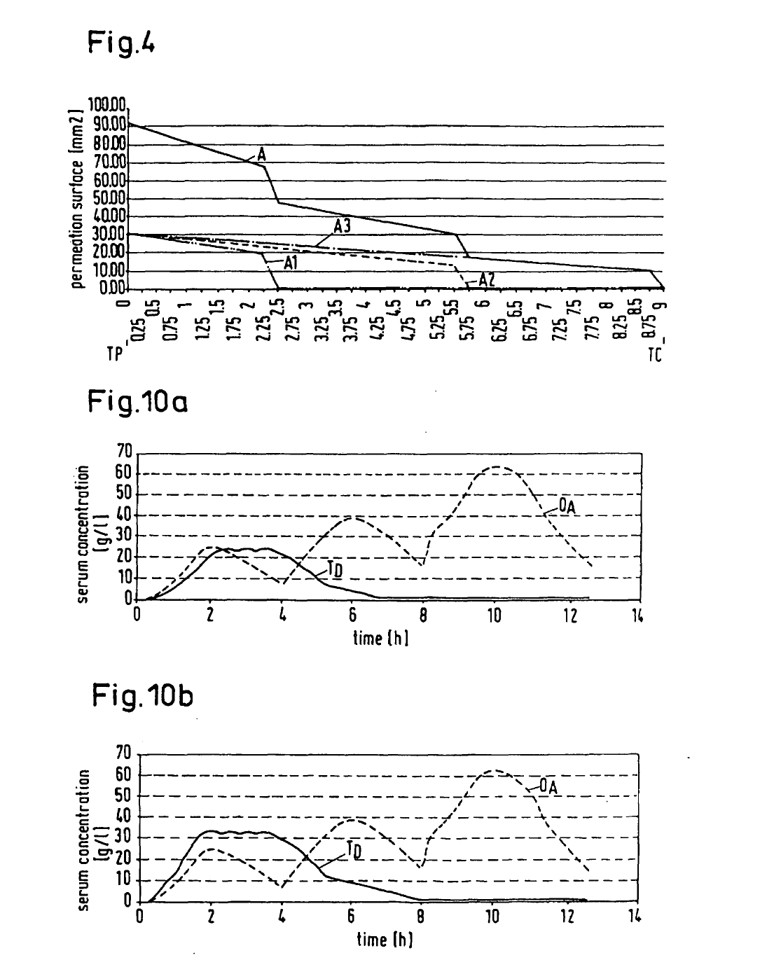

FIG. 4 shows an example of the total permeation surface A as a function of time. FIG. 4 shows the corrected total permeation surface A(t), which is the total permeation surface A(t) of the epidermis 1a only. The laser-porator 10 allows to micro-porating a biological membrane 1 by the creation of an array of micropores 2 in said biological membrane 1, whereby the number of micropores 2 and the shape of these micropores 2 is properly selected so that the sum of the micropores 2 forming an initial permeation surface, and that the permeation surface A (t) of the initial permeation surface decreases in a predetermined function over time, due to cell growth in the micropores 2.

The initial microporation dataset D according to FIG. 4 comprises three groups of cylindrical micropores 2 with different shapes:
a first group consisting of 415 pores with a diameter of 250 μm, a depth of 50 μm and a permeation surface A1 as a function of time.
a second group consisting of 270 pores with a diameter of 250 μm, a depth of 100 μm and a permeation surface A2 as a function of time.
a third group consisting of 200 pores with a diameter of 250 μm, a depth of 150 μm and a permeation surface A3 as a function of time.

The total permeation surface A as a function of time is the sum of all three permeation surfaces A1, A2 and A3.

All individual pores 2i, which means the initial microporation, are created within a very short period of time, for example, within a time range of less than a second, so that beginning with the time of poration TP, the sum of all created pores 2i forming an initial permeation surface of 90 mm², which, due to cell growth, decreases as a function of time. At the time TC all individual pores 2i are closed, which means that the value of the permeation surface A becomes very small or zero.

Depending on the number of pores 2 and their shape, in particular the diameter and depth of the pores 2, the function over time of the total permeation surface A can be varied in a wide range. This makes it clear that the poration of individual pores 2 does not only determine the initial permeation surface, but also the function of the total permeation surface A over time. FIG. 4 shows the total permeation surface A over a time period of 9 days, starting with an initial permeation surface of 90 mm². The permeation surface A decreases within 9 days to a very small value or to zero. Depending on the shape of the individual pores 2, the time period may be much shorter, for example, just 1 day, or even shorter, for example, a few hours.

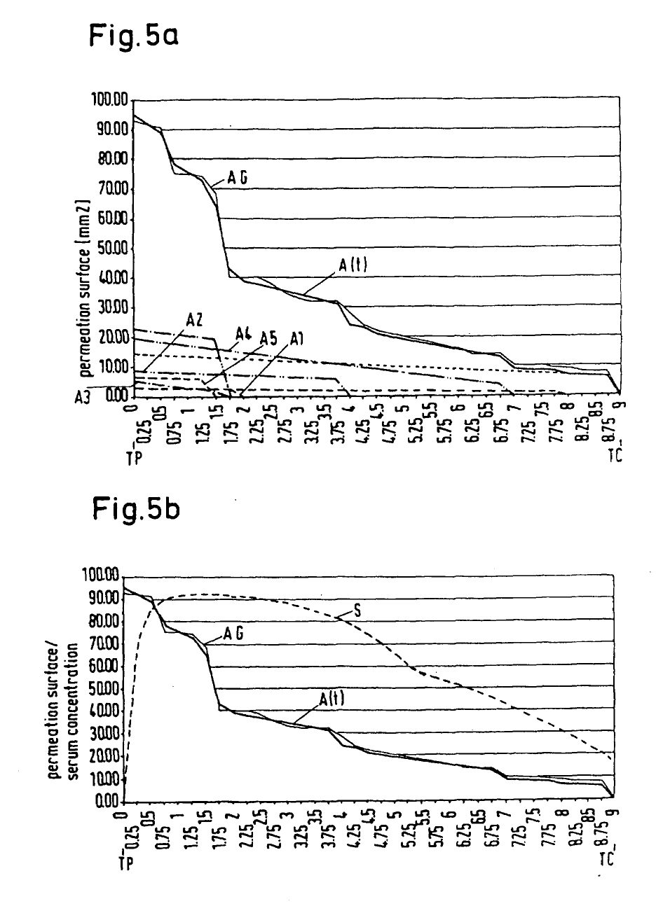

Almost any permeation surface A(t) as a function of time may be establish by a proper selection of the number and the shape of the individual pores 2. FIG. 5a shows a given function A_{G} of a permeation surface as a function of time. FIG. 5a also shows the permeation surface of different groups A1, A2, A3, A4, A5, . . . of individual pores 2 over time. Each group being defined by a number of pores, a diameter and a depth. All individual pores 2 have cylindrical shape. By combining the individual permeation surfaces (A1, A2, A3, A4, A5, . . . ) of all the groups, a permeation surface A(t) is achieved, which function over time is quite similar to the given function A_{G}. The different groups of individual pores, their number and their shape can be determined by mathematical methods known to those skilled in the art. The definition of these groups is stored as the initial microporation dataset D.

FIG. 3c shows a patch 5 containing a drug 5a and being fixed onto the skin 1, above the individual pores 2. FIG. 5b shows the serum concentration S of this drug as a function of time in the blood. The drug is entering the permeation surface by passive diffusion. The amount of drug entering the permeation surface is mainly determined by the permeation surface A(t) over time. Therefore, the serum concentration as a function of time can be determined by an appropriate poration of the skin 1 with an initial microporation.

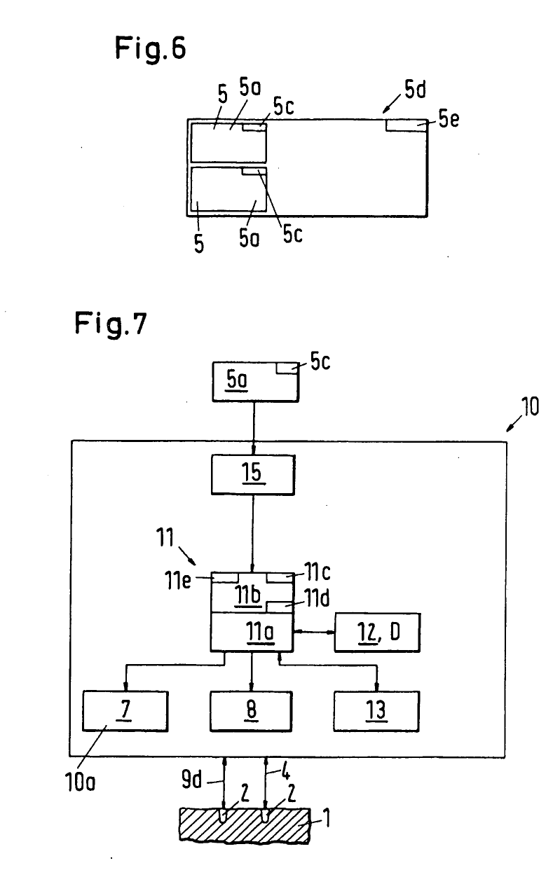

FIG. 6 shows a permeant 5a, which, for example, is a drug or a drug container containing a drug. The permeant 5a comprises permeant information PI stored on a data carrier 5c. A plurality of permeants 5a can be stored in a cassette 5d. The cassette 5d can also comprise a data carrier 5d. The permeant information PI contains at least one data selected from the group: manufacturer ID, product ID, specific product ID, specific drug, drug concentration, nominal drug volume, drug container size, serial number, lot number, expiration date, initial microporation dataset D. The permeant information PI can also comprise information regarding doses, for example a minimal dose/day or a maximal dose/day. The permeant information PI can also contain the information of the entire patient information leaflet.

FIG. 2a shows a micro-porator 10 for porating a biological membrane 1, comprising: a controller 11, an initial microporation dataset D stored in the poration memory 12, and an ablator 10a for creating a microporation on the biological membrane 1, the controller 11 controlling the an ablator 10a based on the initial microporation dataset D, to create the microporation as defined by the initial microporation dataset D. The micro-porator 10 may be programmed with just one fixed initial microporation dataset D. This microporator 10 can, for example, be sold in combination with a specific drug. In a further embodiment, the data carrier 5c, can be inserted into the micro-porator 10, the data carrier 5c containing the initial microporation dataset D.

FIG. 7 shows a micro-porator 10 comprising a controller 11, an interface 15, a poration memory 12, a laser 7, optics 8 and a feedback loop 13. The laser emitting a laser beam 4 to create pores 2 in the skin 1, and the feedback loop 13 emitting a laser beam 9d to measure the depth of the pores 2. The controller 11 contains a poration controller 11a which controls the laser 7 so as to create pores 2 as defined in the poration memory. The controller 11 also contains a main controller 11b which communicates with the poration controller 11a and the interface 15. The interface 15 allows reading at least one parameter selected from the group consisting of: permeant information PI, user information UI, initial microporation dataset D, porator application information PAL The user information UI comprises data such as sex, age, permeants which may or may not be used, maximal or minimal dose, or user ID. The porator application information PAI comprises information about how the porator is used, for example, at which time or date, for which user, for which drug etc. All data mentioned (PI, UI, D, PAI) may be stored on the data carrier 5c of the drug 5a. These data can, for example, be prescribed by a physician.

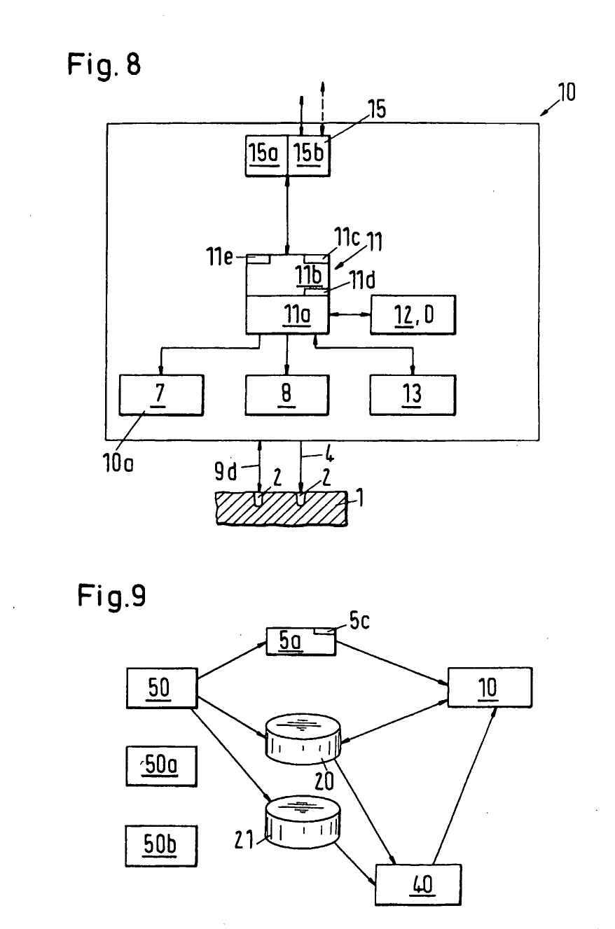

FIG. 8 shows a further micro-porator 10. In contrast to the embodiment disclosed in FIG. 7, the micro-porator 10 according to FIG. 8 has an interface 15 comprising a user-interface 15a to display data and to input data manually, and a data interface 15b to communicate date. The data interface 15b being able to communicate data selected from the group consisting of: 1-D. 2-D and 3-D bar codes, 1-D, 2-D and 3-D symbologies, holograms, written text, radio frequency identification devices (RFIDs), integrated chip smart cards, EEPROMs, magnetic strip, wire and wireless communication.

The controller 11 of the porator 10 can comprise an internal database 20 that stores a plurality of data of at least one parameter selected from the group consisting of: permeant information PL user information UI, initial microporation dataset D, porator application information PAI. The database 20 may for example comprise two different initial microporation datasets D, each dataset defining the application of the same drug but with different speed, as disclosed in FIGS. 10a to 10b. The appropriate initial microporation dataset out of the two initial microporation datasets D may manually be selected, for example, based on the needs of the user.

The controller 11 of the porator 10 may also comprise a selector 11d that automatically selects the most appropriate initial microporation dataset D out of a plurality of initial microporation datasets D. For example several initial microporation datasets D are stored in the internal database 20, taking into account different ages or different weights of users. Based on user information UI (for example age, weight), the most appropriate initial microporation dataset D is selected.

The controller 11 may also comprise an inhibitor 11c which inhibits the porator from porating when at least one of the following conditions is met: user information UI not correct, permeant information PI not correct, no valid initial microporation dataset D, user not allowed to apply the permeant, user not allowed to apply the initial microporation dataset D, user wants to apply the permeant outside a given timeframe (too early, too late), porator not directed onto the biological membrane. This inhibitor 11c allows a safe use of the microporator 10, or avoids a misuse of the microporator 10. The controller 11 can for example be used as a reminder to apply a drug, for example for elderly people who may forget applying an important drug. The controller 11 can be used to prevent suicide or addiction, in that the application of a certain drug is restricted, for example in time, in number or in amount. The controller 11 can be used to prevent the application of a wrong drug. The controller 11 can be used to prevent the application of a drug, for example, when the drug expired or when the drug, for certain reasons, may not be used any more.

The controller 11 of the porator 10 may also comprise a timer (11e) which recalls using the porator if it has not been used within a given period of time.

FIG. 10a to 10b show the administration of the same drug, for example 100 mg acetylsalicylic acid, the drug being arranged on the skin 1 as disclosed in FIG. 3c. Depending on the permeation surface A(t) as a function of time, the level of the serum concentration as well as the time period within which the drug is released, can be predescribed. In FIG. 10a the permeation surface A(t), not shown in detail, is chosen such that the maximal serum concentration is about 25 g/l over a short period of time of about two hours. FIG. 10b shows a fast application (turbo) of the drug, with maximal serum concentration of about 30 g/l over a short period of time of about two hours. One advantage of the invention is, that with transdermal application TD the serum concentration reaches an about constant value, in contrast to oral application OA, which shows a heavy fluctuation. A further advantage is that the same amount of drug, e.g. the same patch, applied onto the skin 1, causes a different serum concentration, depending only on the function of the permeation surface A over time. This allows administering the same drug in different ways. This also allows administering the same drug in an individual way, in that the initial permeation surface is created depending on individual parameters of the person the drug is applied to.

The integrated permeant administering system comprises at least one permeant 5a, data of at least one initial microporation dataset D for the respective permeant 5a, and a micro-porator 10 for porating a biological membrane 1 as defined by the initial microporation dataset D. The micro-porator 10 comprises an interface 15 to read at least one parameter selected from the group consisting of: permeant information PI, initial microporation dataset D, user information UI, porator application information PAL The permeant 5a comprises at least one parameter selected from the group consisting of permeant information PI, initial microporation dataset D. The system can further comprise a database 20 with a plurality of initial microporation datasets Di for the same permeant 5a, the various microporation datasets Di relating to at least one parameter selected from the group consisting of user information UI, amount of permeant absorption, time function of permeant absorption. The system can consisting of a database 20 comprising permeant information PI for a plurality of different permeants, and comprising at least one initial microporation dataset Di for each permeant.

FIG. 9 shows a system comprising an external database 20, with which a plurality of micro-porators 10 can communicate. The micro-porator 10 can read the data carrier 5c of a permeant 5a. For each permeant 5a, at least one initial microporation dataset D is stored in the external database 20, so the porator 10 can get the initial microporation dataset D for every permeant 5a. For the data transfer, for example, a wireless communication is used.

In a preferred embodiment the database 20 is provided and/or updated by the company in charge for the permeant 5a, preferably pharmaceutical companies 50, 50a, 50b. These companies are in a position to provide the required data for combining a permeant 5a, for example a transdermal patch, with an appropriate initial microporation dataset D, to get an effective amount of permeant in the human body.

Also a physician may get access to the database 20 as well as to database 21 containing information regarding the permeant 5a. The physician may tailor an initial microporation dataset D, based on data of the databases 20, 21 and based, for example, on individual needs of a patient, and transfer this initial microporation dataset D to the micro-porator 10.

The method for administering a permeant 5a with a micro-porator 10 comprises the steps of choosing a permeant 5a, getting an initial microporation dataset D for the respective permeant 5a, porating a biological membrane 1 as defined by the initial microporation dataset D, and applying the permeant 5a on the porated biological membrane 1.

This method if further explained by way of examples:

### Example 1

A drug 5a comprises a data carrier 5c with an initial microporation dataset D. This dataset is transferred to the micro-porator, which then creates the micropores. The drug 5a is then applied onto the porated area of the skin.

### Example 2

A drug 5a comprises a data carrier 5c with a plurality of initial microporation datasets D, for example three datasets D, one for slow, medium and fast application of the drug, as disclosed in FIGS. 10a to 10b. The user may through the user interface 15a select the appropriate initial microporation dataset D, according to which the micropores then are created.

### Example 3

A drug 5a comprises at least a specific drug-ID. The porator has access to an inter or external database 20 wherein initial microporation datasets D for a plurality of different drugs 5a are stored. The microporator 10 reads the specific drug-ID and retrieves from the database 20 the corresponding initial microporation dataset D, according to which the micropores then are created. The internal or external database 20 may be updated regularly, for example by data provided by pharmaceutical companies, so that the database 20 contains a library of an initial microporation datasets D for different drugs 5a. The library may contain further data, for example minimal dose/day, maximal dose/day etc. One advantage of this method is that the pharmaceutical company has direct influence to the administration of a drug. This makes the administration of the drug safer and also more efficient.

### Example 4

A drug 5a comprises at least a specific drug-ID. The porator has access to an internal or external database 20 wherein initial microporation datasets D for a plurality of different individual parameters like sex, weight, age or individual restrictions are stored. The microporator 10 reads the specific drug-ID, the microporator 10 reads the individual parameters of the user and then retrieves from the database 20 the corresponding initial microporation dataset D, according to which the micropores then are created.

### Example 5

A drug 5a comprises at least a specific drug-ID. A physician has access to a database 21 of various drugs 5a as well as to an external database 20 containing a lot of initial microporation datasets. Base on these data the physician may create an individual initial microporation dataset D for a specific user. The microporator 10 reads the specific drug-ID, and the microporator 10 reads the individual initial microporation dataset D created by the physician, according to which the micropores then are created.

These were only examples of a wide variety of possibilities about how to administer a permeant like a drug with the integrated permeant administering system according to the invention.

The database 20 can also be arranged within the micro-porator 10. This database 20 can regularly be updated, for example by use of a wireless link like GSM (Global Systems for Mobile Communications), SMS or Bluetooth, by access to the internet, by access to a docking station, for example in a drug store, or by a physical data carrier.

1. A micro-porator (10) for porating a biological membrane (1), comprising:
a) a controller (11)
b) an initial microporation dataset (D);
c) and an ablator (10a) that is configured to create a microporation on the biological membrane (1), wherein the controller (11) is configured to control controlling the ablator (10a) based on the initial microporation dataset (D) to thereby create the microporation as defined by the initial microporation dataset (D).

2. The porator of claim 1, wherein the ablator (10a) is a laser source (7) that is configured to emit a pulsed beam (4) onto various locations to thereby create a microporation comprising of a plurality of individual pores (2).

3. The porator of claim 1, wherein the ablator (10a) comprises at least three electrodes and is configured to apply a voltage between the electrodes in contact with the biological membrane (1) to thereby cause a current to pass within the biological membrane (1) to thereby generate a microporation comprising of at least two micro-channels in the biological membrane (1).

4. The porator of claim 1, comprising an interface (15) that is configured to allow reading at least one parameter selected from the group consisting of: permeant information (PI), user information (UI), initial microporation dataset (D), and porator application information (PAI).

5. The porator of claim 4, wherein the interface (15) comprises a user-interface (15a) to input data manually.

6. The porator of claim 4, wherein the interface (15) comprises a data interface (15b) that is configured to communicate data, selected from the group consisting of: 1-D, 2-D and 3-D bar codes, 1-D, 2-D and 3-D symbologies, holograms, written text, radio frequency identification devices (RFIDs), integrated chip smart cards, EEPROMs, magnetic strip, wire-based communication and wireless communication.

7. The porator of claim 1, wherein the controller (11) comprises an internal database (20a) that is configured to store a plurality of data of at least one parameter selected from the group consisting of: permeant information (PI), user information (UI), initial microporation dataset (D), and porator application information (PAI).

8. The porator of claim 1, wherein the controller (11) comprises a selector (11b) that is configured to allow selection of a desirable selects the most appropriate initial microporation dataset (D) out of a plurality of initial microporation datasets (D).

9. The porator of claim 1, wherein the controller (11) comprises an inhibitor (11a) that is configured to inhibit the porator from porating when at least one condition is met selected from the group consisting of: user information (UI) not correct, permeant information (PI) not correct, no valid initial microporation dataset (D), user not allowed to apply the permeant, user not allowed to apply the initial microporation dataset (D), user wants to apply the permeant outside a predetermined given timeframe, and porator not directed onto the biological membrane.

10. The porator of claim 1, wherein the controller (11) comprises a timer (11c) that is configured to recall using the porator if it has not been used within a given period of time.

11. An integrated permeant administering system, the system comprising:
a) at least one permeant (5a),
b) data of at least one initial microporation dataset (D) for the respective permeant (5a),
a) and a micro-porator (10) that is configured to allow porating a biological membrane (1) as defined by the initial microporation dataset (D).

12. The system of claim 11, wherein the micro-porator (10) comprises an interface (15) that is configured to read at least one parameter selected from the group consisting of: permeant information (PI), initial microporation dataset (D), user information (UI), and porator application information (PAI).

13. The system of claim 11, wherein the permeant (5a) comprises at least one parameter selected from the group consisting of: permeant information (PI), and initial microporation dataset (D).

14. The system of claim 11, further comprising a database (20) comprising a plurality of initial microporation datasets (Di) for the same permeant (5a), the microporation datasets (Di) relating to at least one parameter selected from the group consisting of: user information (UI), amount of permeant absorption, and time function of permeant absorption.

15. The system of claim 11, further comprising a database (20) comprising permeant information (PI) for a plurality of different permeants, and comprising at least one initial microporation dataset (Di) for each permeant.

16. The system of claim 14, wherein the database (20) is an external database (20), and wherein the database is configured such that a plurality of micro-porators (10) can communicate with the same database (20).

17. The system of claim 14, wherein the database (20) is configured to allow updating by a company that provides the permeant.

18. The system of claim 11, wherein the initial microporation dataset (D) is configured to allow prescription by a physician.

19. A permeant for a porator of claim 1, wherein at least one of the permeant, a permeant container and a permeant cassette comprises at least one readable information selected from the group consisting of: permeant information (PI) and initial microporation dataset (D).

20. The permeant of claim 19, further comprising a media with stored information selected from the group consisting of: 1-D, 2-D and 3-D bar codes, 1-D, 2-D and 3-D symbologies, hologram, written text, radio frequency identification device (RFID), integrated chip smart card, EEPROM, and magnetic strip.

21. A method for operating the micro-porator (10) of claim 1, comprising the steps of:
a) providing an initial microporation dataset (D);
b) operating the micro-porator (10) according to the initial microporation dataset (D); and
c) creating a microporation on the biological membrane (1) that corresponds to the microporation as defined by the initial microporation dataset (D).

22. A method for administering a permeant (5a) with a porator according to claim 1, comprising the steps of:
a) choosing a permeant (5a),
b) getting an initial microporation dataset (D) for the respective permeant (5a),
c) porating a biological membrane (1) as defined by the initial microporation dataset (D), and
d) applying the permeant (5a) on the porated biological membrane (1).
